(19) United States
(12) Patent Application Publication
(10) Pub. No.: US 2023/0390278 A1
(43) Pub. Date: Dec. 7, 2023

(54) SYNERGISTIC COMBINATIONS OF VALPROOIC ACID AND QUINACRINE

(71) Applicant: VESTLANDETS INNOVASJONSSELSKAP AS, Bergen (NO)

(72) Inventors: Bjørn Tore GJERTSEN, Bergen (NO); Vibeke ANDRESEN, Bergen (NO); Calum LEITSCH, Bergen (NO)

(21) Appl. No.: 18/031,765

(22) PCT Filed: Oct. 12, 2021
(86) PCT No.: PCT/EP2021/078160

§ 371 Date: Apr. 13, 2023

(30) Foreign Application Priority Data
Oct. 13, 2020 (GB) 2016193.1

(51) Int. Cl. A61K 31/473; A61K 31/19; A61P 35/02; A61K 9/20

(52) U.S. Cl. CPC A61K 31/473; A61K 31/19; A61P 35/02; A61K 9/20

(57) ABSTRACT

The present invention relates to synergistic combinations of quinacrine and valproic acid for the treatment of cancer, preferably acute myeloid leukaemia (AML); and methods of treating cancer using such synergistic combinations.

The present invention relates to synergistic combinations of quinacrine and valproic acid for the treatment of cancer, preferably acute myeloid leukaemia (AML); and methods of treating cancer using such synergistic combinations.

Acute myeloid leukemia (AML) is a cancer of the myeloid line of blood cells, characterized by the rapid growth of abnormal cells that build up in the bone marrow and blood, where they interfere with normal blood cells. Symptoms may include feeling tired, shortness of breath, easy bruising and bleeding, and increased risk of infection. Occasionally, spread may occur to the brain, skin or gums. As an acute leukemia, AML progresses rapidly and is typically fatal within weeks or months, if left untreated.

First-line treatment of AML consists primarily of chemotherapy and is divided into two phases: induction and post-remission (or consolidation) therapy. The goal of induction therapy is to achieve a complete remission by reducing the number of leukemic cells to an undetectable level; the goal of consolidation therapy is to eliminate any residual undetectable disease and achieve a cure. Hematopoietic stem cell transplantation is a pivotal consolidation therapy in high and intermediate risk patients; it is usually considered if induction chemotherapy fails in good risk patients. Re-transplantation is only used occasionally after a person relapses after transplantation.

All FAB classification subtypes except M3 are usually given induction chemotherapy with cytarabine (ara-C) and an anthracycline (most often daunorubicin). This induction chemotherapy regimen is known as “7+3” (or “3+7”), because the cytarabine is given as a continuous IV infusion for seven consecutive days while the anthracycline is given for three consecutive days as an IV push. Up to 70% of people with AML will achieve a remission with this protocol. Other alternative induction regimens, including high-dose cytarabine alone, FLAG-like regimens or investigational agents, may also be used. Because of the toxic effects of therapy, including myelo-suppression and an increased risk of infection, induction chemotherapy may not be offered to the very elderly, and the options may include less intense chemotherapy or palliative care.

The M3 subtype of AML, also known as acute promyelocytic leukemia (APL), is almost universally treated with the drug all-trans-retinoic acid (ATRA) in addition to induction chemotherapy, usually an anthracycline.

Even after complete remission is achieved, leukemic cells likely remain in numbers too small to be detected with current diagnostic techniques. If no further post-remission or consolidation therapy is given, almost all people with AML will eventually relapse. Therefore, more therapy is necessary to eliminate undetectable disease and prevent relapse.

The specific type of post-remission therapy is individualized based on a person's prognostic factors and general health. For good-prognosis leukaemias (i.e. inv(16), t(8;21), and t(15;17)), people will typically undergo an additional three to five courses of intensive chemotherapy, known as consolidation chemotherapy. For people with overt residual disease, at intermediate and at high risk of relapse (e.g. those with high-risk cytogenetics, underlying MDS, or therapy-related AML), allogeneic stem cell transplantation is usually recommended if the person is able to tolerate a transplant and has a suitable donor.

For people who are not eligible for a stem cell transplant, immunotherapy with a combination of histamine dihydrochloride (Ceplene) and interleukin 2 (Proleukin) after the completion of consolidation has been shown to reduce the absolute relapse risk by 14%, translating to a 50% increase in the likelihood of maintained remission.

For people with relapsed AML, the only proven potentially curative therapy is a hematopoietic stem cell transplant, if one has not already been performed. In 2000, the monoclonal antibody-linked cytotoxic agent gemtuzumab ozogamicin (Mylotarg) was approved in the United States for people aged more than 60 years with relapsed AML who are not candidates for high-dose chemotherapy.

For relapsed acute promyelocytic leukemia (APL), arsenic trioxide is approved by the USFDA.

Combination chemotherapy involves treating a patient with two or more different drugs simultaneously. The drugs may differ in their mechanism and side-effects. The biggest advantage of this is that it minimises the chances of developing resistance to any one agent. Furthermore, the drugs can often be used at lower doses, reducing toxicity.

There remains, however, a need for new treatments of AML, particularly for efficacious combination therapies. There is a particular need for new treatments for relapsed and resistant AML, and particularly for treatment of unfit patients and patients with ongoing infections. Given the number of drugs that are known or have been tried for the treatment of AML and similar leukaemias, the number of permutations and combinations of possible drug therapies is clearly very large.

Quinacrine (also known as Mepacrine or by the trade name Atabrine) is a drug with several medical applications; it was originally developed as an anti-malaria drug and used during the Second World War. It is related to chloroquine and mefloquine. The main uses of quinacrine today are as an anti-protozoal, anti-rheumatic and an intrapleural sclerosing agent. Antiprotozoal uses include targeting tapeworm and giardiasis, where quinacrine is indicated as a primary agent for patients with metronidazole-resistant giardiasis and patients who should not receive or cannot tolerate metronidazole. Quinacrine is also used off-label for the treatment of systemic lupus erythematosus (SLE), indicated in the treatment of discoid and subcutaneous lupus erythematosus, particularly in patients unable to take chloroquine derivatives.

As an intrapleural sclerosing agent, it is used as pneumothorax prophylaxis in patients at high risk of recurrence, e.g. cystic fibrosis patients. It is still used in some countries as a non-surgical female sterilization agent and is under evaluation for efficacy against Creutzfeldt-Jakob disease.

Drug screens in AML cell lines and effects on AML patient samples and in pre-clinical xenograft in vivo mouse models have suggested that quinacrine may be useful for the treatment of AML (Eriksson et al., 2015; Eriksson et al., 2017).

Valproate (VPA), and its valproic acid, sodium valproate, and valproate semi-sodium forms, are medications primarily used to treat epilepsy and bipolar disorder and to prevent migraine headaches. They are useful for the prevention of seizures in those with absence seizures, partial seizures, and generalized seizures. There have been reports of the synergistic use of valproic acid with nutlin-3 for the treatment of AML, in vitro and in a xenograft mouse model (McCormack et al., 2012); and of the use of valproic acid with all-trans retinoic acid plus low-dose cytarabine for the treatment of AML patients (Fredly et al., (2013), Clinical Epigenetics 5:13).

It has now been found that combinations of quinacrine and valproic acid demonstrate synergy in the treatment of AML. It is an object of the invention therefore to provide pharmaceutical combinations comprising (A) quinacrine or a pharmaceutically-acceptable salt thereof, and (B) valproic acid or a pharmaceutically-acceptable salt thereof, for the treatment of cancer, preferably AML. It is another object to provide methods of treating cancer using pharmaceutical combinations of the invention.

In one embodiment, the invention provides a pharmaceutical combination comprising:
(A) quinacrine or a pharmaceutically-acceptable salt thereof, and
(B) valproic acid or a pharmaceutically-acceptable salt thereof,

wherein the pharmaceutical combination is in the form of a combined preparation for simultaneous, separate or sequential use, preferably for the treatment of cancer. Preferably, the cancer is acute myeloid leukaemia (AML).

In another embodiment, the invention provides a pharmaceutical combination of the invention for use in therapy or for use as a medicament.

In another embodiment, the invention provides a method of treating cancer in a subject comprising simultaneously, sequentially or separately administering to a subject in need thereof therapeutically-effective amounts of components (A) and (B) of a pharmaceutical combination of the invention.

In another embodiment, the invention provides a pharmaceutical combination of the invention, for use in the treatment of cancer, wherein components (A) and (B) are simultaneously, separately or sequentially administered to the subject for the treatment of the cancer.

In yet another embodiment, the invention provides the use of components (A) and (B) of a pharmaceutical combination of the invention in the manufacture of a pharmaceutical combination for simultaneous, separate or sequential use for the treatment of cancer. Preferably, the cancer is AML. Preferably, the amounts of components (A) and (B) in the pharmaceutical combination are synergistic amounts.

Component (A) of the pharmaceutical combination is quinacrine or a pharmaceutically-acceptable salt thereof. Quinacine (or a pharmaceutically-acceptable salt thereof) may be abbreviated herein as QC. The chemical formula for quinacrine is (RS)-N′-(6-chloro-2-methoxy-acridin-9-yl)-N, N-diethyl-pentane-1,4-diamine. Its structural formula is given below:

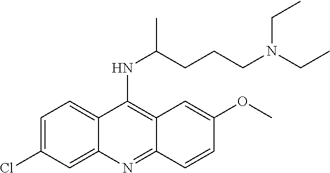 [SMILES: CCN(CC)CCCC(C)Nc1c2ccc(Cl)cc2nc2ccc(OC)cc12]

Quinacrine is a stereo-isomeric compound with one chiral centre. As used herein, quinacrine may be used as a mixture of R- and S-enantiomers (i.e. as the racemate) or in the form of the individual R- or S-enantiomers. In some embodiments, quinacrine is in the form of the R-stereoisomer. Quinacrine is also known as mepacrine and by the trade-name Atabrine®.

Component (B) of the combination is valproic acid or a pharmaceutically-acceptable salt thereof. Valproic acid (or a pharmaceutically-acceptable salt thereof) may be abbreviated herein as VPA. The chemical formula for valproic acid is 2-propylvaleric acid. Its structural formula is given below:

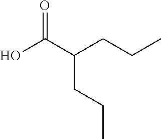

Valproic acid is also commonly known as valproate (VPA, without sodium), sodium valproate (“sodium”), and valproate semi-sodium (“semisodium”). Semi-sodium valproate is a mixture of sodium valproate and valproic acid (without sodium). The invention relates to all of these forms.

Trade names for valproic acid include Absenor (Orion Corporation, Finland); Convulex (G.L. Pharma GmbH, Austria); Depakene (Abbott Laboratories in US and Canada); Depakine (Sanofi Aventis, France); Depakine (Sanofi Synthelabo, Romania); Depalept (Sanofi Aventis, Israel); Deprakine (Sanofi Aventis Finland) Encorate (Sun Pharmaceuticals, India); Epival (Abbott Laboratories, US and Canada); Epilim (Sanofi Synthelabo, Australia and South Africa); Stavzor (Noven Pharmaceuticals Inc.); Valcote (Abbott Laboratories, Argentina); Valpakine (Sanofi Aventis, Brazil); and Orfiril (Desitin Arzneimittel GmbH, Norway).

Preferably, component (B) is present in the pharmaceutical combination as:
(i) sodium valproate,
(ii) valproic acid without sodium, or
(iii) a mixture of (i) and (ii),

or a pharmaceutically-acceptable salt thereof.

Components (A) and/or (B) may independently be in the form of one or more pharmaceutically-acceptable salts. As used herein, the term “pharmaceutically-acceptable salt” refers to a salt that retains the desired biological activity of the parent compound and does not impart any undesired toxicological effects (see, e.g. Berge, S. M., et al. (1977), J. Pharm. Sci. 66:1-19).

Examples of such salts include acid addition salts and base addition salts. Acid addition salts include those derived from non-toxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from non-toxic organic amines, such as N,N′-dibenzylethylenediamine, N-methylglucamine, chloroprocaine, choline, diethanolamine, ethylenediamine, procaine and the like.

Preferably, quinacrine is in the form of quinacrine hydrochloride (or quinacrine dihydrochloride). Preferably, the pharmaceutically-acceptable salt of valproic acid is the sodium salt (Na⁺).

Unless stated otherwise, a structural formula given herein or a chemical name refers to the corresponding compound itself, but also encompasses the tautomers, stereoisomers, optical and geometric isomers (e.g. enantiomers, diastereomers, E/Z isomers, etc.), racemates, mixtures of separate enantiomers in any desired combinations, mixtures of diastereomers, and mixtures of the forms mentioned hereinbefore.

The compounds and salts according to the invention may be present in solvated form (e.g. with pharmaceutically-acceptable solvents such as e.g. water, ethanol etc.) or in unsolvated form. Generally, for the purposes of the present invention the solvated forms, e.g. hydrates, are to be regarded as of equal value to the unsolvated forms.

The pharmaceutical combination of the invention is in the form of a combined preparation for simultaneous, separate or sequential use. Similarly, in the methods of the invention, components (A) and (B) of the pharmaceutical combination may be administered to a patient simultaneously, separately or sequentially.

The term “combined preparation” includes both fixed combinations and non-fixed combinations.

The term “fixed combination” means that the active ingredients (e.g. components (A) and (B)) are in the form of a single entity or dosage unit. In other words, the active ingredients are present in a single composition or formulation.

The term “non-fixed combination” means that the active ingredients (e.g. components (A) and (B)) are present in different entities or dosages (e.g. as separate compositions or formulations), for example as a kit of parts. The independent components (A) and (B) (in their desired compositions or formulations) can then be administered simultaneously, separately or sequentially, at the same time point or at different time points.

Where the administration is simultaneous, components (A) and (B) are administered to the subject at the same time, but not necessarily together. Components (A) and (B) may be present in a single composition or they may be present in different compositions. Components (A) and (B) may be administered at the same site or at different sites (in or on the subject). Components (A) and (B) may be administered by the same route or different routes.

Where the administration is sequential, the delay in administering the second component should not be such as to lose the benefit of the synergistic effect arising from use of the combination. Therefore, in one embodiment sequential treatment involves administration of each component of the combination within a period of 11 days. In another embodiment, this period is 10 days. In another embodiment, this period is 9 days. In another embodiment this period is 8 days. In another embodiment, this period is 7 days. In another embodiment, this period is within 6 days. In another embodiment, this period is within 5 days. In another embodiment, this period is within 4 days. In another embodiment, this period is within 3 days. In another embodiment, this period is within 2 days. In another embodiment, this period is within 24 hours. In another embodiment, this period is within 12 hours.

Components (A) and (B) may each be administered once or at a plurality of times.

Components (A) and (B) may be administered in any order, e.g. component (A) first and then component (B); or component (B) first and then component (A). In some embodiments, Component (A) is administered first to a subject, preferably for 2-4 days, before administration of Component (B). This optimizes the anti-leukaemic effect of the combination and avoids valproic acid-induced proliferative effects on leukaemic stem cells.

Preferably, the combination of components (A) and (B) is a synergistic combination. The skilled person will understand that a synergistic combination is one wherein the effect of the combination is greater than the sum of the effects of the individual components.

Synergistic combinations may also be defined as ones which show in vitro synergism in clinically-obtainable doses.

In vivo and in patients, synergism may be defined as combinations that are able to modify biomarkers that are representative of in vitro synergism. In vivo and in patients, synergism may also be defined as a significantly increased benefit compared to monotherapy (e.g. Student's t test, p<0.01).

Synergism may be quantified using the Chou-Talalay combination index (CI) (see “Evaluation of combination chemotherapy: integration of nonlinear regression, curve shift, isobologram, and combination index analyses”, Zhao L, et al. Clin Cancer Res. (2004) Dec. 1;10(23):7994-8004; and “Computerized quantitation of synergism and antagonism of taxol, topotecan, and cisplatin against human teratocarcinoma cell growth: a rational approach to clinical protocol design”, Chou T C, Motzer R J, Tong Y, Bosl G J., J. Natl. Cancer Inst. (1994) Oct. 19;86(20):1517-24). This combination index (CI) method is based on the multiple drug effect equation derived from the median-effect principle of the mass-action law. This provides a quantitative definition for strong synergism (CI<0.3), synergism (CI=0.3-0.9), additive effect (CI=0.9-1.1) or antagonism/no benefit (CI>1.1), and it provides the algorithm for computer software for automated simulation for drug combinations. It takes into account both the potency (the D(m) value) and the shape of the dose-effect curve (the m value) of each drug alone and their combination. The Chou-Talalay combination index (CI) may be estimated using the Synergy R package (see “Preclinical versus Clinical Drugs Combination Studies”, Chou T C. Leuk. Lymphoma. (2008);49(11):2059-2080, and references therein, all of which are specifically incorporated herein by reference). The CI of the combination may be tested in a suitable cell-line, e.g. the AML cell line MV4-11 (as shown in the Examples).

Preferably, the pharmaceutical combination of the invention is a synergistic combination wherein the Chou-Talalay combination index (CI) is less than 0.9, 0.8, 0.7, 0.6, 0.5, 0.4, or 0.2. In some embodiments, the CI is 0.1-0.2, 0.1-0.3, 0.3-0.5, 0.5-0.7 or 0.7-0.9. In some embodiments, the CI is 0.85, 0.75, 0.73, 0.63, or less than one of the afore-mentioned values.

Components (A) and (B), whether present in a single composition or in separate compositions, may independently be formulated with one or more pharmaceutically-acceptable carriers, excipients and/or diluents.

As used herein, “pharmaceutically acceptable carrier” includes any and all solvents, dispersion media, coatings, antibacterial and antifungal agents, isotonic and absorption delaying agents, and the like that are physiologically-compatible. Preferably, the carrier is suitable for intravenous, intramuscular, subcutaneous, parenteral, spinal or epidermal administration (e.g. by injection or infusion).

Examples of suitable aqueous and non-aqueous carriers that may be employed in the pharmaceutical combinations of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These combinations or parts thereof may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Pharmaceutically-acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

The pharmaceutical combinations of the invention also may include a pharmaceutically acceptable anti-oxidant.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Subjects who are treated with pharmaceutical combinations of the invention may additionally be administered (prior to, simultaneously with, or following administration of a component disclosed herein) with one or more other active agents. Such other active agents may include one or more of the following:
(i) a cytotoxic agent which enhances or augments the therapeutic effect of component (A) and/or (B); and
(ii) a radiotoxic agent which enhances or augments the therapeutic effect of the component (A) and/or (B).

For example, the pharmaceutical combination may additionally comprise one or of the following: hydroxyurea, 6-mercaptopurine, a Bcl-2 inhibitor (e.g. venetoclax) and AXL kinase inhibitor (e.g. bemcentinib).

In some embodiments, the pharmaceutical combination does not comprise one or more of nutlin-3, all-trans-retinoic acid, cytarabine, hydroxyurea or 6-mercaptopurin.

One or both components may be formulated as a solution (e.g. sterile injectable solution), micro-emulsion, liposome, or other ordered structure suitable for a use herein.

Sterile injectable solutions can be prepared by incorporating the component in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated herein. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

Alternatively, one or both components may be administered as sustained-release or controlled-release formulations, in which case less frequent administration is required. Such formulation include implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art (see, e.g. Sustained and Controlled Release Drug Delivery Systems (1978) J. R. Robinson, ed., Marcel Dekker, Inc., N.Y).

Dosage and frequency vary depending on the half-life of the component in the patient. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

Preferably, component (A) is administered in tablet or capsule form. Preferably, component (B) is in the form of a tablet. In some embodiments, component (A) and/or (B) are administered per orally.

The amount of component (A) or component (B) which can be combined with a carrier material to produce a single dosage form will vary depending upon the subject being treated, and the particular mode of administration. It will generally be that amount of the component (A) or (B) which produces a therapeutic effect and, in combination, produces a synergistic therapeutic effect.

Generally, out of 100 per cent, this amount will range from about 0.01 per cent to about 99 per cent of the component, preferably from about 0.1 per cent to about 70 per cent, most preferably from about 1 per cent to about 30 per cent of the component in combination with a pharmaceutically-acceptable carrier.

Dosage regimens are adjusted to provide the optimum desired response (e.g. a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage.

Dosage unit form as used herein refers to physically discrete units suited as unitary dosages for the subjects to be treated; each unit contains a predetermined quantity of the component (A) or (B) calculated to produce the desired therapeutic effect in association with the required pharmaceutical carrier. The specification for the dosage unit forms of the invention are dictated by and directly dependent on (a) the unique characteristics of the component and the particular therapeutic effect to be achieved, and (b) the limitations inherent in the art of compounding such a component for the treatment of sensitivity in individuals.

A “therapeutically-effective dosage” of component (A) or (B) preferably results in a decrease in severity of disease symptoms, an increase in frequency and duration of disease symptom-free periods, or a prevention of impairment or disability due to the disease affliction. For example, for the treatment of AML, a “therapeutically effective dosage” preferably inhibits cancer cell growth or tumour growth by at least about 20%, at least about 30%, more preferably by at least about 40%, at least about 50% even more preferably by at least about 60%, at least about 70% and still more preferably by at least about 80% or at least about 90%, relative to untreated subjects. The ability of a component to inhibit tumour growth can be evaluated in an animal model system predictive of efficacy in human tumours. Alternatively, this property of a component can be evaluated by examining the ability of the compound to inhibit cell growth, such inhibition can be measured in vitro by assays known to the skilled practitioner. A therapeutically-effective amount of a therapeutic compound can decrease tumour size, or otherwise ameliorate symptoms of AML in a subject. One of ordinary skill in the art would be able to determine such amounts based on such factors as the subject's size, the severity of the subject's symptoms, and the particular composition or route of administration selected.

For administration of component (A) or component (B), the dosage ranges from about 0.0001 to 100 mg/kg, for example, 0.001 to 50 mg/kg, 0.005 to 20 mg/kg, 0.01 to 10 mg/kg and more usually 0.01 to 5 mg/kg, of the host body weight of each component. For example dosages can be 0.05 mg/kg body weight, 0.1 mg/kg body weight, 0.3 mg/kg body weight, 0.3 mg/kg body weight, 0.5 mg/kg body weight, 1 mg/kg body weight, 2 mg/kg body weight, 3 mg/kg body weight, 4 mg/kg body weight, 5 mg/kg body weight 6 mg/kg body weight, 7 mg/kg body weight, 8 mg/kg body weight, 9 mg/kg body weight, 10 mg/kg body weight, 12 mg/kg body weight, 15 mg/kg body weight, 20 mg/kg body weight, 25 mg/kg body weight, 30 mg/kg body weight, or within the range of 0.1-20 mg/kg, 0.5-15 mg/kg, 1-10 mg/kg, 2-8 mg/kg, 3-7 mg/kg, 4-6 mg/kg.

Preferably, the dosage of components (A) and (B) are fixed and are not relative to body weight. Preferably, the dosage of component (A) is 50-500 mg/day, more preferably 100-400 mg/day. Preferably, the dosage of component (B) is 200-4000 mg/day, e.g. 400-600 mg/day.

In some embodiments, the dosage of component (A) is 50-150, preferably about 100 mg/kg/day, which is preferably administered intra-peritoneally. In some embodiments, the dosage of component (B) is 300-400, preferably about 350 mg/kg/day, which is preferably administered orally.

In some methods, dosage of each component is independently adjusted to achieve a plasma concentration of about 100 ng/ml-1000 μg/ml, 1-750 μg /ml, 10-600 μg/ml, 15-500 μg/ml, 20-400 μg/ml and in some methods about 25-300 μg/ml.

The plasma concentration may be the blood plasma concentration or bone marrow plasma concentration. Quinacrine is known to accumulate in certain tissues, including white blood cells; the concentration in the relevant tissue will therefore be significantly higher than in plasma. Plasma concentrations may be determined by liquid chromatography-mass spectrometry (LC-MS).

In some preferred embodiments, the desired patient plasma concentration of Component (A) (e.g. quinacrine) is up to 150 nM or up to 300 nM. For example, the desired patient plasma concentration of Component (A) may be 10-50 nM, 50-100 nM, 100-150 nM, 150-200 nM, 200-250 nM or 250-300 nM. For example, the desired patient plasma concentration of Component (A) may be 50-100 nM, 100-200 nM or 200-300 nM.

In some preferred embodiments, the desired patient plasma concentration of Component (B) (e.g. valproic acid) is up to 800 μM, e.g. 300-700 μM. For example, the desired patient plasma concentration of Component (B) may be 10-50 μM, 50-100 μM, 100-150 μM, 150-200 μM, 200-250 μM, 250-300 μM, 300-350 μM, 350-400 μM, 400-450 μM, 450-500 μM, 500-550 μM, 550-600 μM, 600-650 μM, 650-700 μM, 700-750 μM or 750-800 μM. For example, the desired patient plasma concentration of Component (B) may be 50-100 μM, 100-200 μM, 200-300 μM, 300-400 μM, 400-500 μM, 500-600 μM, 600-700 μM or 700-800 μM.

In some embodiments, the desired patient plasma concentration of Component (A) is 10-50 nM; and the desired patient plasma concentration of Component (B) is 50-100 μM, 100-200 μM, 200-300 μM, 300-400 μM, 400-500 μM, 500-600 μM, 600-700 μM or 700-800 μM. In some embodiments, the desired patient plasma concentration of Component (A) is 50-100 nM; and the desired patient plasma concentration of Component (B) is 50-100 μM, 100-200 μM, 200-300 μM, 300-400 μM, 400-500 μM, 500-600 μM, 600-700 μM or 700-800 μM. In some embodiments, the desired patient plasma concentration of Component (A) is 100-150 nM; and the desired patient plasma concentration of Component (B) is 50-100 μM, 100-200 μM, 200-300 μM, 300-400 μM, 400-500 μM, 500-600 μM, 600-700 μM or 700-800 μM. In some embodiments, the desired patient plasma concentration of Component (A) is 150-200 nM; and the desired patient plasma concentration of Component (B) is 50-100 μM, 100-200 μM, 200-300 μM, 300-400 μM, 400-500 μM, 500-600 μM, 600-700 μM or 700-800 μM. In some embodiments, the desired patient plasma concentration of Component (A) is 200-250 nM; and the desired patient plasma concentration of Component (B) is 50-100 μM, 100-200 μM, 200-300 μM, 300-400 μM, 400-500 μM, 500-600 μM, 600-700 μM or 700-800 μM. In some embodiments, the desired patient plasma concentration of Component (A) is 250-300 nM; and the desired patient plasma concentration of Component (B) is 50-100 μM, 100-200 μM, 200-300 μM, 300-400 μM, 400-500 μM, 500-600 μM, 600-700 μM or 700-800 μM.

An exemplary treatment regime entails administration of component (A) and/or component (B) once per day, once every 2 days, once per week, once every two weeks, once every three weeks, once every four weeks, once a month, once every 6 weeks, once every 3 months or once every three to 6 months.

A further exemplary treatment regime entails first administering component (A) before (e.g. on each of 1, 2, 3 or 4 days before) the first administration of component (B). The first dose of component (A) may be a high dose, for example, 800-1000 mg component (A), followed by lower doses, e.g. 100-400 mg, of component (A). The lower dose of component (A) may be 300 mg per day (e.g. 100 mg morning, midday and evening).

The ratio of the total amounts of component (A) to component (B) to be administered in the combined preparation can be varied, e.g. in order to cope with the needs of a patient sub-population to be treated or the needs of the single patient which different needs can be due to age, sex, body weight, etc. of the patients.

In one embodiment, the ratio of component (A) to component (B) is 1:250 —1:1000, preferably about 1:500 (e.g. 1 μM component (A) with 500 μM component (B)). In another embodiment, the ratio of component (A) to component (B) is 1:2000-1:5000.

Also within the scope of the present invention are kits comprising components (A) and (B), optionally together with instructions for use. The kits may further comprise one or more of the following: (i) an immunosuppressive reagent, (ii) a cytotoxic agent, (iii) a radiotoxic agent, and (iv) a pharmaceutically-acceptable excipient, carrier or diluent.

Also provided is a pharmaceutical combination of the invention for use in therapy or for use as a medicament.

In a further embodiment, the invention provides a method of treating cancer in a subject comprising simultaneously, sequentially or separately administering to a subject in need thereof therapeutically-effective synergistic amounts of components (A) and (B) of a pharmaceutical combination of the invention.

Also provided is a pharmaceutical combination of the invention for use in the treatment of cancer, wherein synergistic amounts of components (A) and (B) are simultaneously, separately or sequentially administered to the patient for the treatment of the cancer.

Also provided is the use of synergistic amounts of components (A) and (B) of a pharmaceutical combination of the invention in the manufacture of a pharmaceutical combination for simultaneous, separate or sequential use for the treatment of cancer.

Preferably, the cancer is AML. In some embodiments, the AML is relapsed AML or drug-resistant AML.

As used herein, the term “subject” is intended to include human and non-human animals. Non-human animals include all vertebrates, e.g. mammals and non-mammals, such as non-human primates, sheep, dogs, cats, cows, horses, chickens, amphibians, and reptiles. Preferably, the subject is a human. Preferred subjects include human patients having AML.

In some embodiments, the patient is one who has a favorable, intermediate or adverse risk for AML. This risk may be established according to cytogenetics and mutational status. In some embodiments, the patient is one who has an infection (e.g. a viral or bacterial infection).

A pharmaceutical combination of the present invention may be administered via one or more routes of administration using one or more of a variety of methods known in the art. Components (A) and (B) may be administered by the same route or by different routes. As will be appreciated by the skilled artisan, the route and/or mode of administration will vary depending upon the desired results. Preferred routes of administration for components (A) and (B) of the invention independently include intravenous, intramuscular, intradermal, intraperitoneal, subcutaneous, spinal or other parenteral routes of administration, for example by injection or infusion.

The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Alternatively, components (A) and (B) may be administered via a non-parenteral route, such as a topical, epidermal or mucosal route of administration, for example, intranasally, orally, vaginally, rectally, sublingually or topically.

Preferably, component (A) and (B) are both administered orally. Preferably, component (A) is administered orally, in tablet or capsule form. Preferably, component (B) is administered orally, in tablet form. Most preferably, component (A) is administered orally in tablet or capsule form and component (B) is administered orally in tablet form.

The disclosure of each reference set forth herein is specifically incorporated herein by reference in its entirety.

### BRIEF DESCRIPTION OF THE FIGURES

FIG. 1 (A) shows the capacity of quinacrine and valproic acid to induce apoptosis in the human AML cell line, MV4-11, both as monotherapies and when combined at a constant ratio. FIG. 1 (B) illustrates the combination index values as determined by the Chou-Talalay method for determining synergism and expressed as fraction affected (fa) 0-1.0.

FIG. 2A demonstrates synergy after treatment of MV4-11 cells for 72 hours with QC (2 μM) and VPA (0.6 mM) as compared to either treatments alone or untreated cells as evaluated by Hoechst (DNA) staining and scored for normal compared to abnormal (apoptotic) nuclei.

FIG. 2B shows MV4-11 cells which have been treated as in FIG. 2A for 24 hours before being lysed and analyzed by immunoblotting for the expression of ribosomal protein S6 (RSP6) and microtubule-associated proteins 1A/1B light chain 3B (LC3B) where actin was used as a loading control.

FIG. 2C shows electron microscopy images of MV4-11 cells treated as in FIG. 2B.

FIG. 3 (A) shows the capacity of quinacrine and valproic acid to induce apoptosis in primary AML blasts, both as monotherapies and in combination.

FIG. 3 (B) shows the synergistic capacity of quinacrine and valproic acid to induce apoptosis in primary AML cells as determined by bliss independence analysis.

FIG. 4 (A) shows the capacity of quinacrine and valproic acid to repress MV4-11 tumour growth in vivo in a subcutaneous murine model of AML, both as monotherapies and in combination.

FIG. 4 (B) shows the weights (g) of the mice from start of treatment to endpoint.

FIG. 5. Patient 1. A-B. Protein expression and subcellular localization in blood cells from Patient 1 before and after in vivo treatment with QC and VPA.

FIG. 5 (A). Immunoblotting for expression of RSP6 and LC3B, where HSP90 is included as a loading control.

FIG. 5 (B)-(C). Ex vivo bone marrow and blood samples from Patient 1 (before start of treatment) treated ex vivo with QC and VPA evaluated by WST-1.

FIG. 6. Patient 2. Protein expression in bone marrow and blood cells from Patient 1 before and after in vivo treatment with QC and VPA.

FIG. 6 (A). Immunoblotting for LC3B, where GAPDH is included as a loading control.

FIG. 6 (B). Ex vivo bone marrow (7% blast (microscopy), 17% blasts flow cytometry) from Patient 2 (before start of treatment) treated ex vivo with QC and VPA evaluated by WST-1.

### EXAMPLES

The present invention is further illustrated by the following Examples, in which parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1: Synergistic Capacity of QC and VPA in AML Cell Line

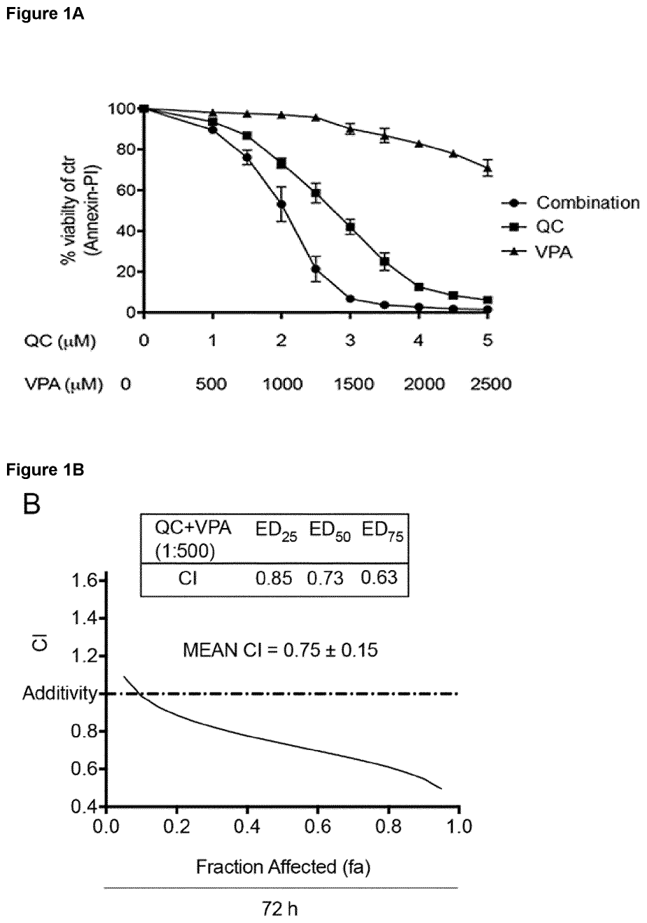

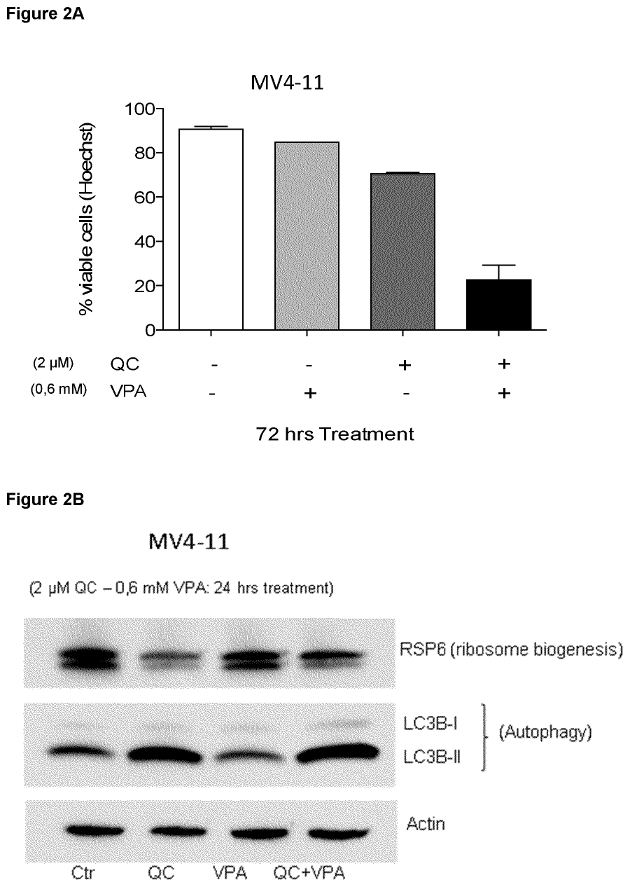

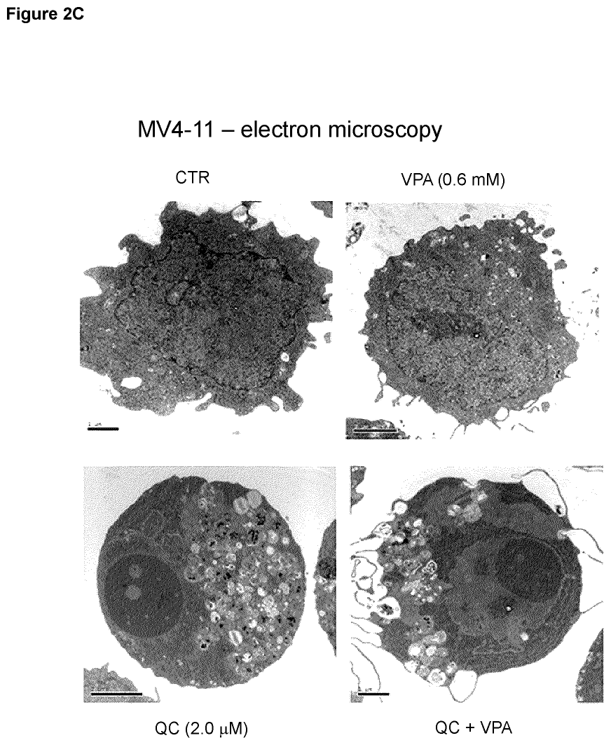

To determine the synergistic capacity of QC and VPA to induce apoptosis in the AML cell line, MV4-11, the experiment illustrated in FIG. 1 was performed. Flow cytometry assessment of Annexin V and propidium iodide (PI) staining of cells exposed to QC and VPA enabled the assessment of cell death and apoptosis. Cells were treated with both agents alone and in combination, and concentrations were maintained at a fixed ratio of 1:500 (QC:VPA). VPA exposure comprised 72 hours, whilst QC treatment was added during the final 24 hrs. Combination index (CI) values were then calculated according to the Chou-Talalay method; the effect on cell viability was expressed as fraction of cells affected (fa). The results are shown in FIGS. 2A-2C.

The experiment shown in FIG. 2A demonstrates synergy after treatment of MV4-11 cells for 72 hours with QC (2 μM) and VPA (0.6 mM) as compared to either treatments alone or untreated cells as evaluated by Hoechst (DNA) staining and scored for normal compared to abnormal (apoptotic) nuclei. In FIG. 2B, MV4-11 cells have been treated as in FIG. 2A for 24 hours before being lysed and analyzed by immunoblotting for the expression of ribosomal protein S6 (RSP6) and microtubule-associated proteins 1A/1B light chain 3B (LC3B) where actin was used as a loading control. FIG. 2C shows electron microscopy images of MV4-11 cells treated as in FIG. 2B.

### Example 2: Synergistic Capacity of QC and VPA in Primary AML Cells

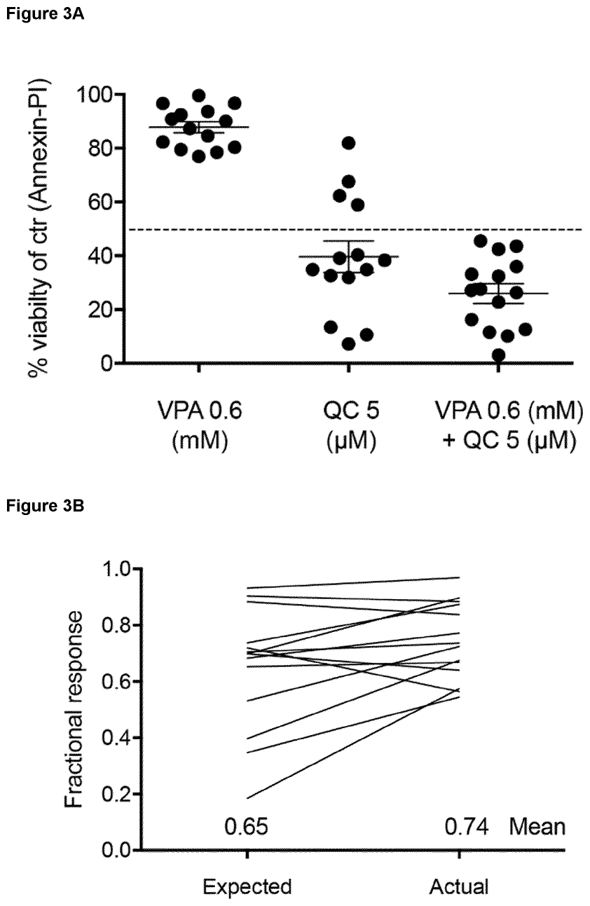

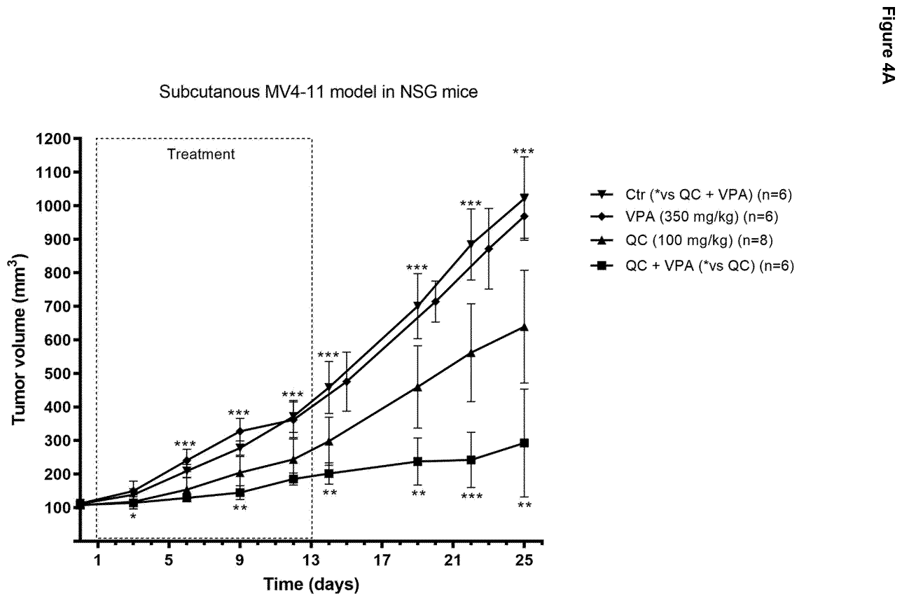

To determine the synergistic capacity of QC and VPA to induce apoptosis in primary AML cells isolated from the peripheral blood of patients, the experiment illustrated in FIG. 3 was performed. As shown in FIG. 3A, primary cells from 14 different patients were exposed to QC (2 μM) and VPA (0.6 mM) both alone and in combination for a period of 24 hrs. Flow cytometry assessment of Annexin V and propidium iodide (PI) staining of treated cells enabled assessment of cell death and apoptosis. Patients exhibited a range of sensitivities to the agents both alone and when combined. The ability of the compounds to synergize in the treatment of primary AML cells was evaluated using a bliss independence analysis. FIG. 3B shows a comparison of the grouped means of the expected and actual fractional responses; a general trend of synergistic response to the therapies was seen.

### Example 3: Synergistic Capacity of QC and VPA in Vivo

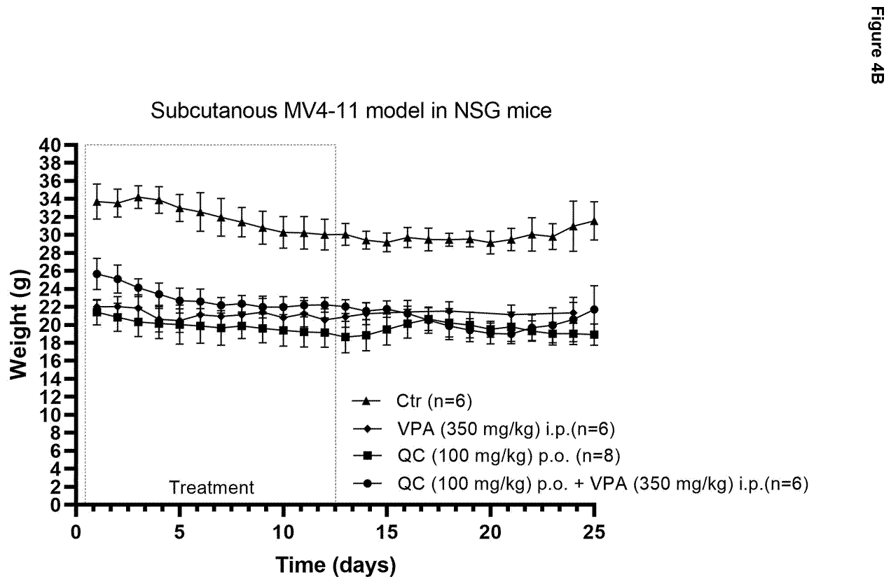

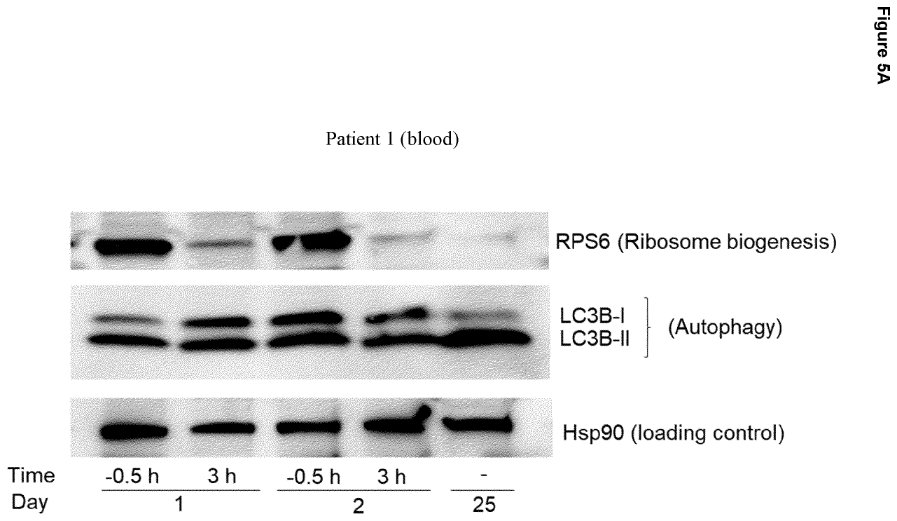

To determine the synergistic capacity of QC and VPA in vivo, the experiment illustrated in FIG. 4A was performed. 26 NOD-scid/IL-2Rγnull mice containing subcutaneous MV4-11 cell tumours were divided into 4 groups: 1 receiving saline as a control, 1 QC only, 1 VPA only and one with both drugs in combination. QC (100 mg/kg/day) was administered per oral and VPA (350 mg/kg/day) via intraperitoneal injection. The mice were treated daily for 13 days; weights were recorded daily and tumour volumes were recorded every third day until the experimental endpoint (tumour volume 1000 mm³) was reached and mice were sacrificed. Asterisks are indicative of statistical significance using two-tailed Student's T test (*p<0.05, ** p<0.01, *** p <0.001). Error bars=SD. The results are shown in FIGS. 4B-4C.

### Example 4: Single Separate Patients Treated Off-Label With QC and VPA or QC Only

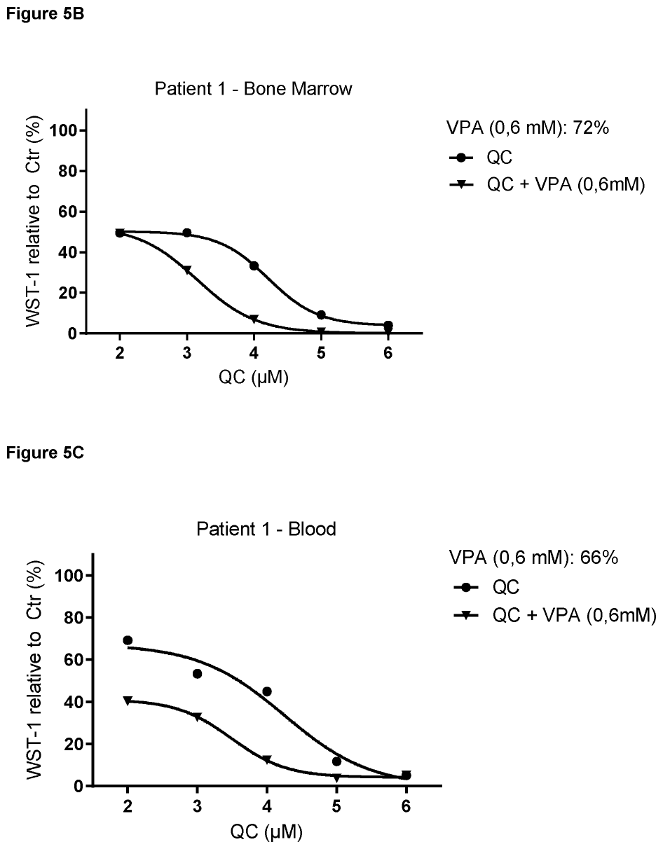

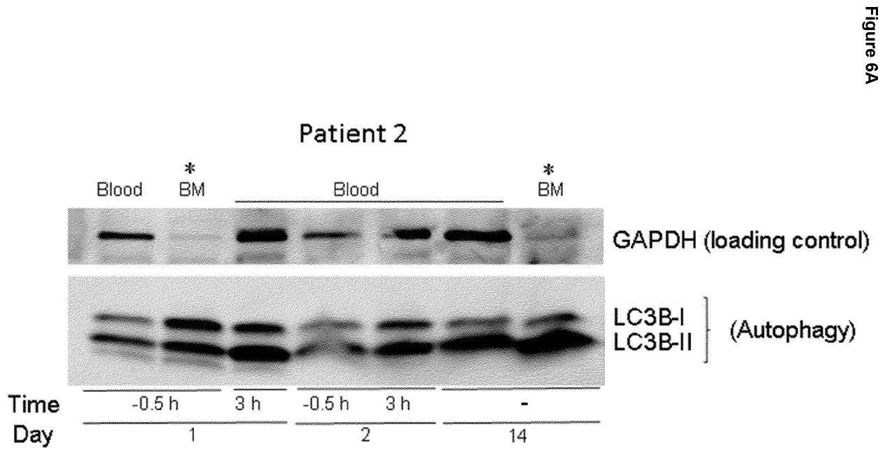

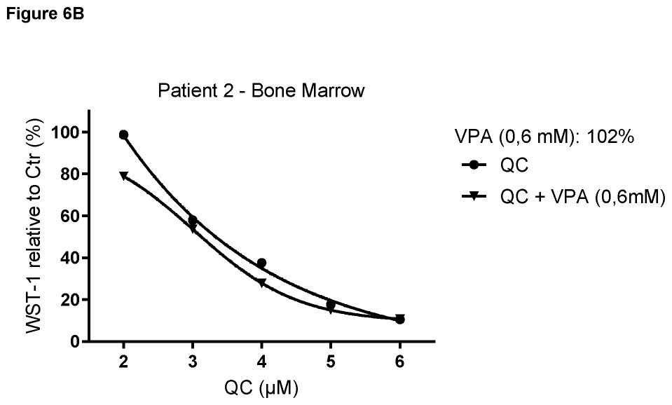

Four AML patients (Table 1) were single separately treated off-label with QC and VPA and one patient with QC only (see Tables 2-6 for dosing and plasma concentrations of QC and VPA as measured by LC/MS). The results are shown in FIGS. 5-6.

TABLE 1
Overview of single separately off-label treated patients

| Patient | Age | Sex | Diagnosis |
|---|---|---|---|
| 1 | 71 | M | Relapsed secondary AML from MDS |
| 2 | 75 | F | Secondary AML from secondary CMML-2 |
| | | | from follicular lymphoma |
| 3 | 76 | M | Relapsed Refractory AML |
| 4 | 55 | M | Relapsed Refractory secondary AML |
| | | | from MDS alloHSCT |
| 5 | 69 | F | Relapsed Refractory AML |

TABLE 2
Patient 1: Overview of treatment period and plasma levels of QC and VPA
in blood and bone marrow where the patient took QC and VPA tablets
every day. Dosage increases and other treatments are as indicated.
Patient 1

| Treatment | | | | | |
|---|---|---|---|---|---|
| Time | QC | VPA | Plasma | | Remarks/ |
| (day) | (mg/day) | (mg/day) | QC (nM) | VPA (μM) | comments |
| 1 | 100 | 300 + 300 | 4 | 17.12 | 3 h after |
| | | | | | intake |
| 5 | 100 | 300 + 600 | | | |
| 8 | 200 | 300 + 600 | | | |
| 10 | 200 | 300 + 600 | | | |
| 15 | 200 | 600 + 1200 | | | |
| 25 | 200 | 600 + 1200 | 52 | 43.04 | |
| 28-35 | HU 500 × 2 | 6MP 50 | | | |
| 36 | 200 | 600 + 1200 | | | |
| 53 | 200 | 600 + 900 | | | |
| 58 | 300 | 600 + 900 | | | |
| 64 | 300 | 600 + 900 | 138 | 25.58 | |
| 99 | 400 | 600 + 900 | | | |
| 128 | 400 | 600 + 900 | 38 | neg | |
| 136 | 400 | 600 + 900 | | | |
| 147 | 400 | 300 + 600 | | | |
| 163 | 400 | 300 + 600 | 38 | 118.86 | |
| | | | BM: 44 | BM:126 | |

| | |
|---|---|
| 169 | MORS |

Neg: negative, BM: bone marrow, QC tablets: 100 mg, VPA tablets: 150 mg, 300 mg and 600 mg, + between dosing indicates dosage morning and evening, HU: hydroxyurea, 6MP: mercaptopurine.

TABLE 3
Patient 2: Overview of treatment period and plasma levels of QC
and VPA in blood and bone marrow (BM) where the patient took QC
and VPA tablets every day. Dosage increases are as indicated.
Patient 2

| Treatment | | | | | |
|---|---|---|---|---|---|
| Time | QC | VPA | Plasma | | Remarks/ |
| (day) | (mg/day) | (mg/day) | QC (nM)) | VPA (μM) | comments |
| 1 | 100 | 300 + 600 | 8 | 156.26 | 3 h after |
| | | | | | intake |
| 15 | 200 | 600 + 600 | 18 | 442.04 | |
| | | | BM: 32 | BM: 329.48 | |
| 37 | 300 | 300 + 900 | | | |
| 49 | 200 | 300 + 600 | | | |
| 65 | 200 | 300 + 600 | 130 | 341.4 | |
| | | | BM: 332 | BM: 280.68 | |

| | |
|---|---|
| 67 | EOT |

EOT: end of treatment. QC tablets: 100 mg, VPA tablets: 150 mg, 300 mg and 600 mg, + between dosing indicates dosage morning and evening.

TABLE 4
Patient 3: Overview of treatment period and plasma levels
of QC and VPA in blood where the patient took QC and VPA
tablets every day. Dosage increases are as indicated.
Patient 3

| Treatment | | | Plasma | | |
|---|---|---|---|---|---|
| Time | QC | VPA | QC | VPA | Remarks/ |
| (day) | (mg/day) | (mg/day) | (nM) | (μM) | comments |
| 1 | 100 | 300 + 300 | 8 | 120.42 | 3 h after |
| | | | | | intake |
| 5 | 100 | 300 + 600 | | | |
| 8 | 200 | 300 + 600 | | | |
| 15 | 300 | 300 + 600 | 300 | 41.26 | |

| | |
|---|---|
| 22 | EOT |
| 23 | MORS |

EOT: end of treatment, QC tablets: 100 mg, VPA tablets: 150 mg, 300 mg and 600 mg, + between dosing indicates dosage morning and evening.

TABLE 5
Patient 4: Overview of treatment period where the patient took
QC and VPA tablets every day. Dosage increases are as indicated.
Patient 4

| Treatment | | | |
|---|---|---|---|
| Time | QC | VPA | Plasma |

| | | | | |
|---|---|---|---|---|
| (day) | (mg/day) | (mg/day) | QC | VPA |

| | | |
|---|---|---|
| 1 | 300 | Not yet analyzed |

| | | |
|---|---|---|
| 6 | 300 | 1200 + 1200 |
| 9 | 300 | 300 + 300 |
| 14 | 300 | 300 + 600 |

| | |
|---|---|
| 18 | EOT |
| 37 | MORS |

EOT: end of treatment, QC tablets: 100 mg, VPA tablets: 150 mg, 300 mg and 600 mg, + between dosing indicates dosage morning and evening.

TABLE 6
Patient 5: Overview of treatment period where the patient took QC tablets
every day. Dosage increases and other treatments are as indicated.
Patient 5

| Time (day) | | Treatment |
|---|---|---|
| 1-5 | | HU 1000 × 2 |
| 10-16 | | HU 500 × 2 |
| | Treatment | |

| | | | |
|---|---|---|---|
| Time | QC | VPA | Plasma |

| | | | | |
|---|---|---|---|---|
| (day) | (mg/day) | (mg/day) | QC | VPA |

| | | | |
|---|---|---|---|
| 22 | 100 | — | Not yetanalyzed |

| | | |
|---|---|---|
| 27 | 200 or 300? | — |

| | |
|---|---|
| 30 | EOT |
| 33 | MORS |

EOT: end of treatment, QC tablets: 100 mg, HU: hydroxyurea.

### REFERENCES

Eriksson et al., (2015), Blood Cancer Journal 5, e307.

Eriksson et al., (2017), Leukemia Research 63: 41-46

Fredly et al., (2013), Clinical Epigenetics 5:13

McCormack et al., (2012), Leukemia 26, 910-917

1. A pharmaceutical combination comprising:
(A) quinacrine or a pharmaceutically-acceptable salt thereof, and
(B) valproic acid or a pharmaceutically-acceptable salt thereof, wherein the pharmaceutical combination is in the form of a combined preparation for simultaneous, separate or sequential use.

2. The pharmaceutical combination as claimed in claim 1, wherein:
(i) component (A) is quinacrine hydrochloride; and/or
(ii) component (B) is the sodium salt of valproic acid.

3. The pharmaceutical combination as claimed in claim 1, wherein components (A) and (B), whether present in a single composition or in separate compositions, are independently formulated with one or more pharmaceutically-acceptable carriers, excipients and/or diluents.

4. The pharmaceutical combination as claimed in claim 1, wherein:
(i) component (A) is in tablet or capsule form; and/or
(ii) component (B) is in tablet form.

5. The pharmaceutical combination as claimed in claim 1, wherein the pharmaceutical combination is in the form of a combined preparation for simultaneous, separate or sequential use for the treatment of cancer.

6. A kit comprising:
(A) quinacrine or a pharmaceutically-acceptable salt thereof; and
(B) valproic acid or a pharmaceutically-acceptable salt thereof.

7. (canceled)

8. A method of treating cancer in a subject comprising simultaneously, sequentially or separately administering to a subject in need thereof therapeutically-effective amounts of components (A) and (B) of a pharmaceutical combination as defined in claim 1.

9-10. (canceled)

11. The method as claimed in claim 8, wherein the cancer is acute myeloid leukaemia (AML).

12. The method as claimed in claim 8, wherein:
(i) the dosage of component (A) is adjusted to achieve a plasma concentration in the subject of 50-150 nM, 150-200 nM, 200-250 nM or 250-300 nM; and/or
(ii) the dosage of component (B) is adjusted to achieve a plasma concentration in the subject of 300-700 μM.

13. The method as claimed in claim 8, wherein component (A) is first administered to the subject before component (B) is first administered to the subject.

14. The pharmaceutical combination as claimed in claim 5, wherein the cancer is acute myeloid leukaemia (AML).

15. The method as claimed in claim 13, wherein component (A) is first administered to the subject on each of 1, 2, 3, or 4 days before component (B) is first administered to the subject.
